(19) United States
(12) Patent Application Publication
(10) Pub. No.: US 2023/0414160 A1
(43) Pub. Date: Dec. 28, 2023

(54) METHODS AND SYSTEMS FOR MEASURING RENAL NEURAL ELECTRICAL ACTIVITY BY ELECTRICALLY STIMULATING IN ABDOMINAL AORTA AND SENSING EVOKED NEURAL ELECTRICAL RESONSE IN RENAL ARTERY

(71) Applicant: Otsuka Medical Devices Co., Ltd., Tokyo (JP)

(72) Inventors: Liang Zhai, Belmont, CA (US); Andrew WU, Los Altos Hills, CA (US); Neil Barman, Menlo Park, CA (US)

(21) Appl. No.: 18/340,241

(22) Filed: Jun. 23, 2023

Related U.S. Application Data
(60) Provisional application No. 63/367,117, filed on Jun. 27, 2022.

(51) Int. Cl. A61B 5/388; A61B 5/00

(52) U.S. Cl. CPC A61B 5/388; A61B 5/6852

(57) ABSTRACT

Techniques for evaluating neural electrical activity of renal nerves of a patient for which a renal denervation procedure has been or is going to be performed are described. A distal portion of a guide catheter is inserted through an abdominal aorta so the distal portion is positioned adjacent to a renal artery ostium, or in a proximal portion of the renal artery. The guide catheter is used to insert a distal portion of a mapping catheter within the renal artery, and a stimulation electrode positioned in the abdominal aorta (within a specified distance of the renal artery ostium) is used to deliver electrical stimulation pulse(s) to evoke a neural electrical response from renal nerves in tissue surrounding the renal artery. A sense electrode positioned in the renal artery, downstream from the stimulation electrode, is used to sense the neural electrical activity evoked in response to the stimulation pulse(s).

### PRIORITY CLAIM

The present application claims priority to U.S. Provisional Patent Application No. 63/367,117, filed Jun. 27, 2022, titled METHODS AND SYSTEMS FOR MEASURING RENAL NEURAL ACTIVITY BY STIMULATING IN ABDOMINAL AORTA AND SENSING EVOKED NEURAL RESPONSE IN RENAL ARTERY which is incorporated herein by reference in its entirety to provide continuity of disclosure.

### FIELD

Embodiments of the present technology generally relate to techniques for use in evaluating neural electrical activity of renal nerves in tissue surrounding a renal artery of a patient for which a renal denervation procedure has been or is going to be performed, and related systems and methods.

### BACKGROUND

The human body's nervous system includes both the somatic nervous system that provides sense of the environment (vision, skin sensation, etc.) and regulation of the skeletal muscles, and is largely under voluntary control, and the autonomic nervous system, which serves mainly to regulate the activity of the internal organs and adapt them to the body's current needs, and which is largely not under voluntary control. The autonomic nervous system involves both afferent or sensory nerve fibers that can mechanically and chemically sense the state of an organ, and efferent fibers that convey the central nervous system's response (sometimes called a reflex arc) to the sensed state information. In some cases, the somatic nervous system is also influenced, such as to cause vomiting or coughing in response to a sensed condition.

Regulation of the human body's organs can therefore be somewhat characterized and controlled by monitoring and affecting the nerve reflex arc that causes organ activity. For example, the renal nerves leading to a kidney can often cause a greater reflexive reaction than desired, contributing significantly to hypertension. Measurement of the nerve activity near the kidney, and subsequent ablation of renal nerves can therefore be used to control the nervous system's overstimulation of the kidney, improving operation of the kidney and the body as a whole.

Because proper operation of the nervous system is therefore an important part of proper organ function, it is desired to be able to monitor and change nervous system function in the human body to characterize and correct nervous system regulation of internal human organs.

New medical therapies have been practiced whereby a catheter is inserted into the body to a specified anatomical location and destructive means are conveyed to nerves by means of the catheter (aka probe) to irreversibly damage tissue in the nearby regions. The objective is to modulate (e.g., abolish) nerve function in the specified anatomic location. The result is that abnormally functioning physiological processes can be terminated or modulated back into a normal range. Unfortunately, such medical therapies are not always successful because of an inability to assess that the neural activity has been successfully abolished. An alternative objective can be to increase a physiologic process or modulate it to an abnormal range.

An example is renal nerve ablation, which is also known to as renal denervation, to relieve hypertension. Various studies have confirmed the relationship of renal nerve activity with blood pressure regulation. In various renal ablation procedures, a catheter is introduced into a hypertensive patient's arterial vascular system and advanced into the renal artery. Renal nerves are located in the arterial wall and/or in regions adjacent to the artery. Destructive means are delivered proximate to the renal artery wall to an extent intended to cause destruction of renal nerve activity. Destructive means include energy such as radiofrequency (RF), microwave, cryotherapy, ultrasound, laser or chemical agents. The objective is to abolish the renal nerve activity. Such nerve activity is an important factor in the creation and/or maintenance of hypertension and abolishment of the nerve activity reduces blood pressure and/or medication burden.

Unfortunately not all patients respond to this therapy. Renal nerve ablation procedures are often ineffective, potentially due to a poor probe/tissue interface. Accordingly, insufficient quantities of destructive means are delivered to the nerve fibers transmitting along the renal artery. One reason is that the delivery of destructive means to the arterial wall does not have a feedback mechanism to assess efficacy of the destruction of the nerve activity. As a consequence an insufficient quantity of destructive means is delivered and nervous activity is not abolished. Clinicians, therefore, would benefit from improved ways to monitor the integrity of the nerve fibers passing through the arterial wall in order to confirm destruction of nerve activity prior to terminating therapy. Current technology for the destruction of nerve activity does not provide practitioners with a feedback mechanism to detect when the desired nervous activity destruction is accomplished. Nerve destructive means are applied empirically without knowledge that the desired effect has been achieved.

It is known that ablation of the renal nerves, with sufficient energy, is able to effect a reduction in both systolic and diastolic blood pressure. Current methods are said to be, from an engineering perspective, open loop; i.e., the methods used to effect renal denervation do not employ any way of measuring, in an acute clinical setting, the results of applied ablation energies. It is only after application of such energies and a period of time (3-12 months) that the effects of the procedure are known.

The two major components of the autonomic nervous system (ANS) are the sympathetic and the parasympathetic nerves. The standard means for monitoring autonomic nerve activity in situations such as described is to insert very small electrodes into the nerve body or adjacent to it. The nerve activity creates an electrical signal in the electrodes which is communicated to a monitoring means such that a clinician can assess nerve activity. This practice is called microneurography and its practical application is by inserting the electrodes transcutaneously to the desired anatomical location. This is not possible in the case of the ablation of many autonomic nerves proximate to arteries, such as the renal artery, because the arteries and nerves are located within the abdomen and cannot be accessed transcutaneously with any reliability. Thus, the autonomic nerve activity cannot be assessed in a practical or efficacious manner.

Deficiencies in the use of existing therapeutic protocols in denervation of autonomic nerves proximate arteries include: 1) The inability to determine the appropriate lesion sites along the artery that correspond to the location of nerves; 2) The inability to verify that the destructive devices are appropriately positioned adjacent to the arterial wall, normalizing the tissue/device interface and enabling energy transfer through the vessel wall; and 3) The inability to provide feedback to the clinician intraoperatively to describe lesion completeness or the integrity of the affected nerve fibers. As a consequence, autonomic nerve ablation procedures are typically performed in a ‘blind’ fashion; the clinician performing the procedure does not know where the nerves are located; and further, whether the nerves have truly been ablated. Instead, surrogates such as calf muscle sympathetic activity (MSNA) or catecholamine spillover into the circulating blood have been used to attempt to evaluate the reduction in organ specific autonomic activity such as renal nerve activity. It is entirely likely that this deficiency could at least partly be responsible for the current variability in clinical responses coming from clinical trials. Therefore, methods and systems that enable determinations with precision of whether a denervation procedure was successful is desirable.

### SUMMARY

Certain embodiments of the present technology are directed to methods for use in evaluating neural electrical activity of renal nerves in tissue surrounding a renal artery of a patient for which a renal denervation procedure has been or is going to be performed. Such a method can include inserting a distal portion of a guide catheter through an abdominal aorta of a patient such that the distal portion of the guide catheter is positioned adjacent to a renal artery ostium located along a lateral aspect of the abdominal aorta, or such that the distal portion of the guide catheter is inserted through the renal artery ostium and into a proximal portion of the renal artery of the patient. The method also includes using the guide catheter to insert a distal portion of a mapping catheter through a distal most end of the guide catheter such that the distal portion of the mapping catheter is positioned within the renal artery of the patient. While the distal portion of the mapping catheter is positioned within the renal artery of the patient, the method also includes using a stimulation electrode positioned in the abdominal aorta within a specified distance (e.g., within 10 millimeters (mm)) of the renal artery ostium to deliver one or more electrical stimulation pulses to thereby evoke a neural electrical response from at least some of the renal nerves in tissue surrounding the renal artery, wherein the stimulation electrode is located on the mapping catheter, on the distal portion of the guide catheter, or on a guidewire. The method also includes using a sense electrode positioned in the renal artery, downstream from the stimulation electrode, to sense the neural electrical activity evoked in response to the one or more electrical stimulation pulses, wherein the sense electrode is located on the mapping catheter. In accordance with certain such embodiments, the sense electrode, which is used to sense the neural electrical activity evoked in response to the one or more electrical stimulation pulses, is positioned downstream of the stimulation electrode by at least 1.5 centimeters (cm), preferably by at least 2.0 cm, and even more preferably by at least 3.0 cm. Additionally, the method includes using the sensed neural electrical activity, evoked in response to the one or more electrical stimulation pulses, as a pre-denervation or post-denervation measurement associated with a renal denervation procedure.

In accordance with certain embodiments, the one or more electrical stimulation pulses are delivered between the stimulation electrode and a return electrode that is also positioned in the abdominal aorta within the specified distance (e.g., 10 mm) of the renal artery ostium. In other embodiments, the one or more electrical stimulation pulses are delivered between the stimulation electrode and a return surface electrode that is positioned on skin of the patient.

In accordance with certain embodiments, the stimulation electrode comprises a deployable stimulation electrode that is deployed such that an outer circumference of the deployable stimulation electrode is in contact with an inner wall of the abdominal aorta within the specified distance (e.g., 10 mm) of the renal artery ostium. In certain such embodiments, the sense electrode comprises a deployable sense electrode that is deployed such that an outer circumference of the deployable sense electrode is in contact with an inner wall of the renal artery, downstream of the deployable stimulation electrode. The deployable sense electrode can be used together with another sense electrode to sense the neural electrical response, evoked in response to the one or more electrical stimulation pulses, wherein the other sense electrode is also located on the mapping catheter downstream of the stimulation electrode. In other embodiments, the deployable sense electrode is used together with another sense electrode to sense the neural electrical response, evoked in response to the one or more electrical stimulation pulses, wherein the other sense electrode is a surface electrode that is positioned on skin of the patient. In accordance with certain such embodiments, the sense electrode, which is used to sense the neural electrical activity evoked in response to the one or more electrical stimulation pulses, is positioned downstream of the stimulation electrode by at least 1.5 cm, preferably by at least 2.0 cm, and even more preferably by at least 3.0 cm.

In specific embodiments, while the distal portion of the mapping catheter is positioned within the renal artery of the patient, the method includes using a stimulation electrode positioned in the abdominal aorta to deliver one or more electrical stimulation pulses to an aorticorenal ganglia thereby evoking a neural electrical response from at least some of the renal nerves in tissue surrounding the renal artery, wherein the stimulation electrode is located on the mapping catheter, on the distal portion of the guide catheter, or on a guidewire. Such a method also includes using a sense electrode positioned in the renal artery, downstream from the stimulation electrode, to sense the neural electrical activity evoked in response to the one or more electrical stimulation pulses, wherein the sense electrode is located on the mapping catheter. The method similarly also includes using the sensed neural electrical activity, evoked in response to the one or more electrical stimulation pulses, as a pre-denervation or post-denervation measurement associated with a renal denervation procedure.

This summary is not intended to be a complete description of the embodiments of the present technology. Other features and advantages of the embodiments of the present technology will appear from the following description in which the preferred embodiments have been set forth in detail, in conjunction with the accompanying drawings and claims.

### BRIEF DESCRIPTION OF THE FIGURES

FIG. 1 illustrates a kidney that receives blood via the abdominal aorta, a renal artery, and branch vessels.

FIG. 2 is a cross-section of a renal artery and is used to describe the various layers and nerves of the renal artery wall.

FIG. 3A shows a distal portion of a guide catheter inserted, via a femoral approach, through the abdominal aorta of a patient such that the distal portion of the guide catheter is positioned adjacent to the renal artery ostium located along a lateral aspect of the abdominal aorta, and also shows a distal portion of a mapping catheter extending through a distal end of the guide catheter and located within the renal artery.

In FIG. 3B shows a distal portion of a guide catheter inserted, via a femoral approach, through the abdominal aorta of a patient such that the distal portion of the guide catheter is inserted through the renal artery ostium and into a proximal portion of the renal artery, and also shows a distal portion of a mapping catheter extending through a distal end of the guide catheter and located within the renal artery.

FIGS. 4A and 4B are high level flow diagrams used to summarize methods according to certain embodiments of the present technology.

FIG. 5A shows an example catheter with its two selectively deployable electrodes in their non-deployed positions.

FIG. 5B shows the catheter, which was introduced in FIG. 5A, with its two selectively deployable electrodes in their deployed positions.

FIG. 6 is a schematic diagram of an example system, according to an embodiment of the present technology, for interfacing with a patient's arterial nerves.

### DETAILED DESCRIPTION

In the following detailed description of example embodiments, reference is made to specific example embodiments by way of drawings and illustrations. These examples are described in sufficient detail to enable those skilled in the art to practice what is described, and serve to illustrate how elements of these examples may be applied to various purposes or embodiments. Other embodiments exist, and logical, mechanical, electrical, and other changes may be made. Features or limitations of various embodiments described herein, however important to the example embodiments in which they are incorporated, do not limit other embodiments, and any reference to the elements, operation, and application of the examples serve only to define these example embodiments. Features or elements shown in various examples described herein can be combined in ways other than shown in the examples, and any such combination is explicitly contemplated to be within the scope of the examples presented here. The following detailed description does not, therefore, limit the scope of what is claimed.

Regulating operation of the nervous system to characterize nerve signaling and modulate organ function includes in some examples introduction of a catheter (aka probe) into the body to a specified anatomical location, and partially destroying or ablating nerves using the probe to destroy nerve tissue in the region near the probe. By reducing nerve function in the selected location, an abnormally functioning physiological process can often be regulated back into a normal range. It would also be possible to modulate nerve function to purposely cause an abnormally functioning that is beneficial to the patient.

Unfortunately, it is typically very difficult to estimate the degree to which nerve activity has been reduced, which makes it difficult to perform a procedure where it is desired to ablate all nerves, or to ablate some, but not all, nerves to bring the nervous system response back into a desired range without destroying the nervous system response entirely. A denervation procedure may be used, for example, to perform renal nerve ablation to treat hypertension, as was noted above.

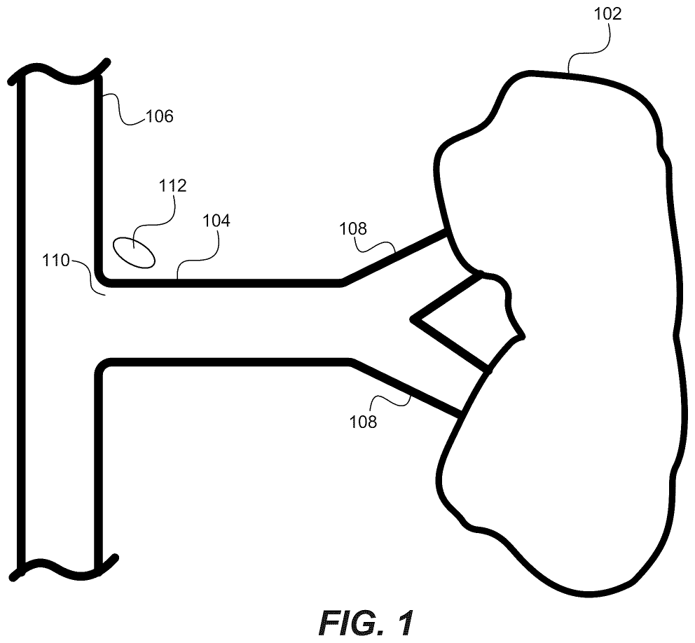

As illustrated in FIG. 1, a kidney 102 receives blood via the abdominal aorta 106, a renal artery 104, and branch vessels 108. FIG. 1 also shows a renal artery ostium 110 located along a lateral aspect of the abdominal aorta 106, wherein the renal artery ostium 110 is the orifice of the branch off the abdominal aorta 106 that opens into the renal artery 104.

Renal sympathetic nerves, which can also be referred to as sympathetic renal nerves, generally follow the abdominal aorta 106 and the renal artery 104 allowing for communication between the brain and the kidney 102. The sympathetic renal nerves include both the afferent sensory renal nerves that carry neural impulses from the kidney 102 to the brain, and the efferent sympathetic renal nerves that carry neural impulses from the brain to the kidney 102. In other words, efferent renal nerves that follow the renal artery 104 carry impulses from the brain to the kidney 102. By contrast, afferent renal nerves that follow the renal artery carry impulses from the kidney 102 to the brain.

Also shown in FIG. 1 is the aorticorenal ganglion 112 which is located near the renal artery ostium 110, between thoracic splanchnic nerves (not shown) that originate in the spinal cord. Postganglionic axons (not shown) then form the renal plexus, which is a dense network of nerve fibers which runs alongside the renal artery 104 and enters the hilum of the kidney 102. A ganglion is typically known as a mass of tissue formed by ganglion cells. The aorticorenal ganglion 112 includes ganglia that provide relay points and intermediary connections between the peripheral and central nervous systems.

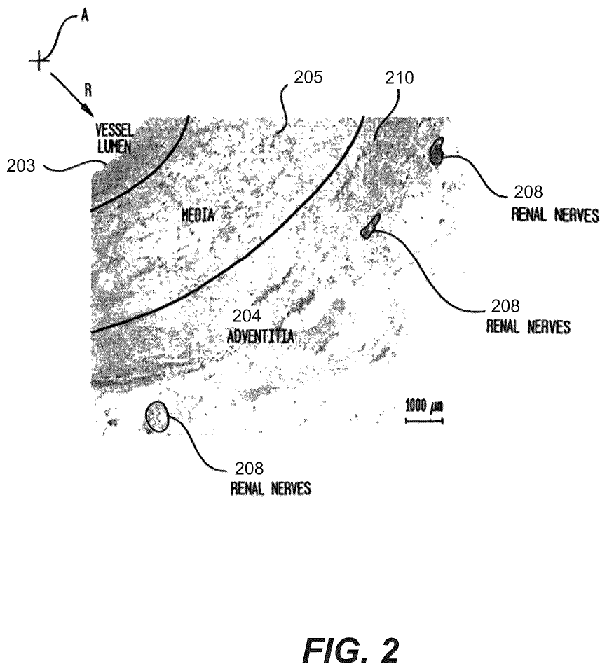

As illustrated in FIG. 2, which is a cross-section of the renal artery 104, the renal artery wall is made up of multiple layers, including: the intima 203, which includes an inner single layer of endothelial cells; the media 205, which is in the center of the artery wall; and the aventitia 204, which is the outside layer. Also shown are the renal nerves 208 that lie within the aventitia 204, on the surface of the renal artery 104, and adjacent to the renal artery 104. The renal nerves 208 surround the renal artery 104. Different individuals have renal nerves 208 in different locations around the renal artery. Thus, the renal nerves may be at different radial distances R from the central axis A of the renal artery 104, and also may be at different locations around the circumference C of the renal artery 104. It is not practical to locate the renal nerves by referring to anatomical landmarks. Moreover, it is difficult or impossible to locate individual renal nerves using common in vivo imaging technology.

As explained above in the Background, renal nerve ablation (aka renal denervation) can be used to treat hypertension. Various studies have confirmed that renal nerve activity has been associated with hypertension, and that ablation of the renal nerves can improve renal function and reduce hypertension. In a typical procedure, a catheter is introduced into a hypertensive patient's arterial vascular system and advanced into the renal artery 104. In a renal denervation procedure, renal nerves 208 located in the arterial wall and in regions adjacent to the renal artery 104 are ablated by a destructive means such as radio frequency (RF) energy, microwave energy, ultrasound energy, cryotherapy, laser or chemical agents to limit the renal nerve activity, thereby reducing hypertension in the patient. The destructive means that is used to perform such a renal denervation procedure can be a transducer that is located on a distal portion of a catheter that is inserted into the renal artery 104. For much of the remaining discussion, it will be assumed that the transducer is an ultrasound transducer that can be activated to deliver unfocused ultrasonic energy radially outwardly so as to suitably heat, and thus treat, tissue within the target anatomical region surrounding the renal artery 104. Such a transducer can be activated at a frequency, duration, and energy level suitable for treating the targeted tissue. In one nonlimiting example, the unfocused ultrasonic energy generated by the transducer may target select nerve tissue of the subject, and may heat such tissue in such a manner as to neuromodulate (e.g., fully or partially ablate, necrose, or stimulate) the nerve tissue. One of skill in the art will recognize that other mechanisms may be used to denervate the nerve tissue. Non limiting examples of mechanisms used to perform a renal denervation procedure include RF, microwave, chemical, cryotherapy, and/or pulsed electric field, but is not limited thereto.

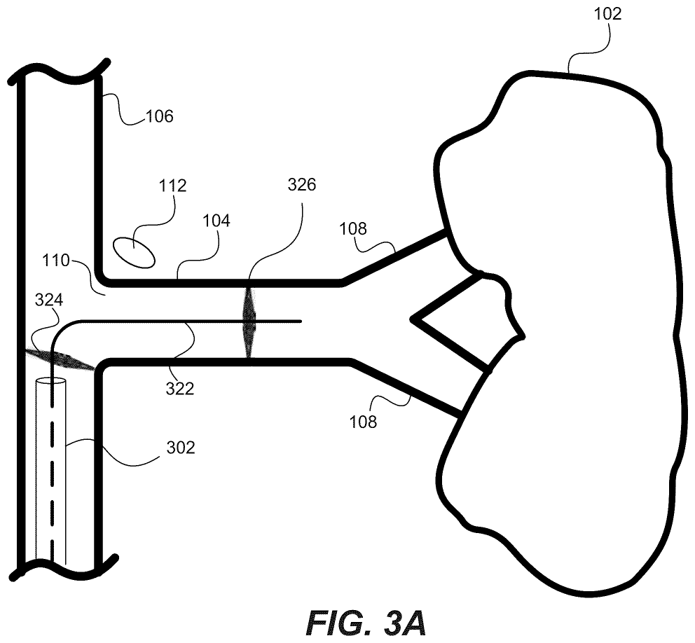

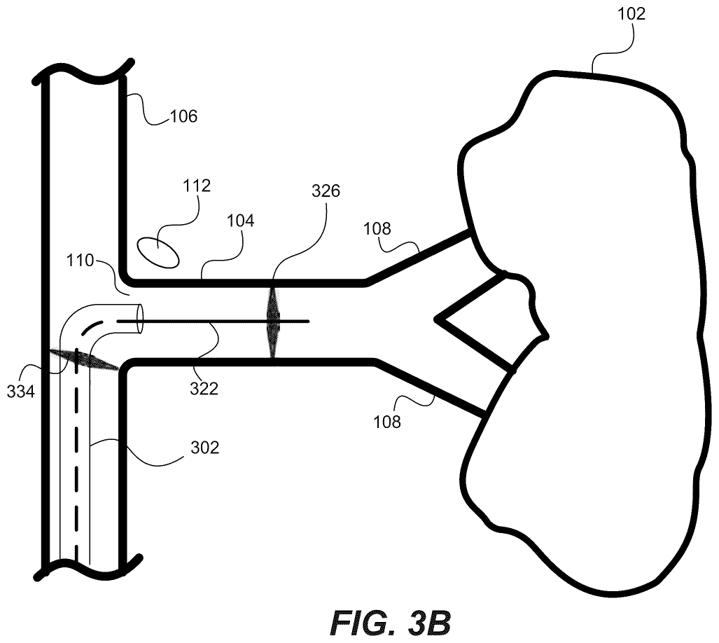

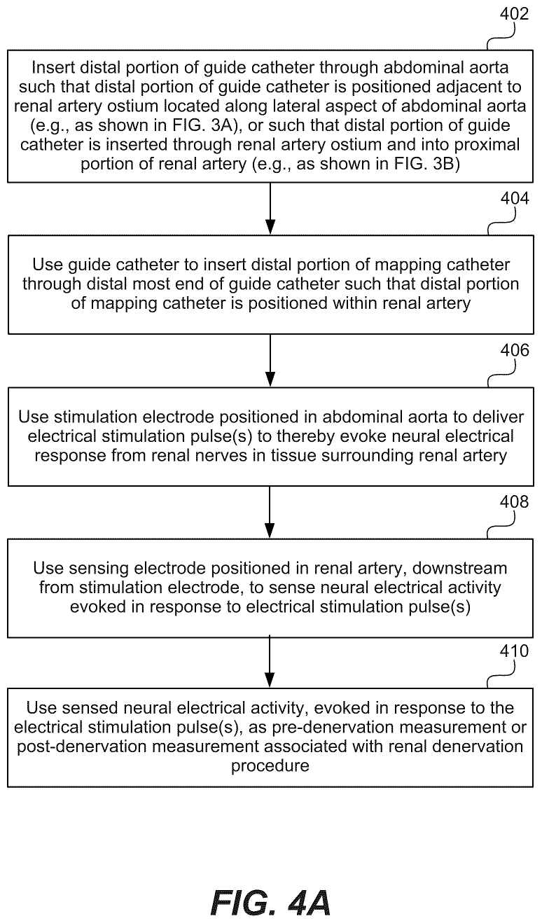

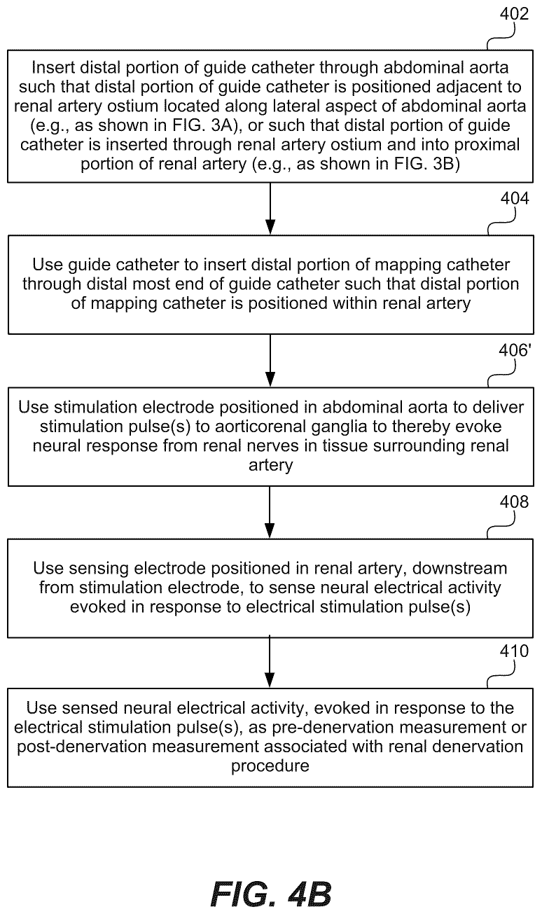

Reference is now made to FIGS. 3A and 3B, which are similar to FIG. 1 in that they show the kidney 102 that receives blood via the abdominal aorta 106, the renal artery 104, and the branch vessels 108, which are labeled the same in FIGS. 3A and 3B as they were in FIG. 1. FIGS. 3A and 3B also show the renal artery ostium 110, which is the orifice of the branch off the abdominal aorta 106 that opens into the renal artery 104. Additionally, FIGS. 3A and 3B show the aorticorenal ganglion 112.

FIG. 3A also shows a distal portion of a guide catheter 302 that is inserted, via a femoral approach, through the abdominal aorta 106 of a patient such that the distal portion of the guide catheter 302 is positioned adjacent to the renal artery ostium 110 located along a lateral aspect of the abdominal aorta 106. In FIG. 3B the distal portion of the guide catheter 302 is shown as being inserted through the renal artery ostium 110 and into a proximal portion of the renal artery 104.

In the embodiments shown in FIGS. 3A and 3B, the guide catheter 302 is used to insert a distal portion of a mapping catheter 322 through abdominal aorta 106, and through the renal artery ostium 110, and into the renal artery 104. While not specifically shown in FIGS. 3A and 3B, a guidewire (not shown) can also be used to help guide the distal portion of the mapping catheter 322 into the renal artery 104. More specifically, after the distal portion of the guide catheter 302 is positioned adjacent to the renal artery ostium 110 (as shown in FIG. 3A) or is positioned in a proximal portion of the renal artery 104 (as shown in FIG. 3B), a guidewire can then be inserted through the guide catheter 302, through the renal artery ostium 110, and into the renal artery 104. The mapping catheter 322 can then be placed over the guidewire by threading a proximal end of the guidewire through a distal end of a guidewire lumen of the mapping catheter 322. The mapping catheter 322 can then be guided into the renal artery 104, over the guidewire and through the guide catheter 302. Instead of using a femoral approach to insert catheters into the abdominal aorta and the renal artery, a radial approach could alternatively be used, as would be appreciated by one of skill in the art.

In accordance with certain embodiments of the present technology, the mapping catheter 322 includes one or more electrodes that is/are used to sense an evoked neural electrical response to electrical stimulation pulses that are delivered using one or more other electrodes of the mapping catheter 322 and/or electrical stimulation pulses that are delivered using one or more other electrodes of the guide catheter 302. The one or more electrodes of the mapping catheter 322 (or the guide catheter 302) that are used to deliver electrical stimulation pulses, which electrical stimulation pulses are intended to evoke a neural response by renal nerves in tissue surrounding the renal artery, are often referred to herein as stimulation electrodes. The one or more electrodes of the mapping catheter 322 that are used to sense the evoked neural electrical response by renal nerves, to the electrical stimulation pulses, are often referred to herein as sense electrodes. It is noted that catheters and/or circuitry coupled thereto (e.g., of an electronic control unit) can be designed such that switching circuitry (e.g., relays and/or transistor switches) can be used to control which one or more electrodes is/are configured as stimulation electrodes and which one or more electrodes is/are configured as sense electrodes, such that at one point in time a specific electrode can be configured as a stimulation electrode, and at another point in time the same specific electrode can be configured as a sense electrode.

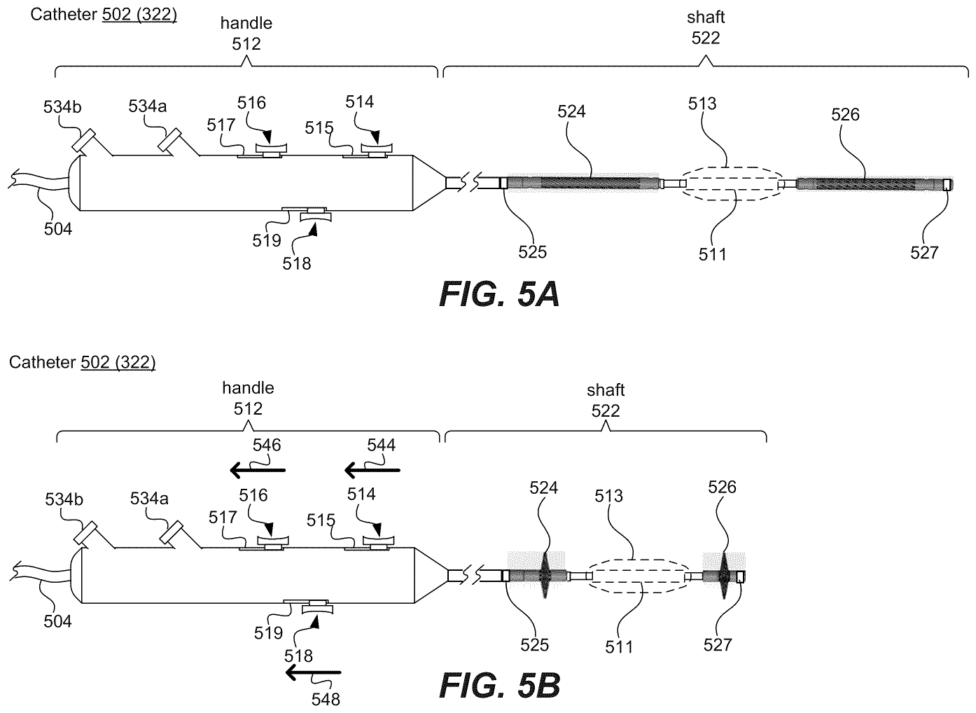

In FIG. 3A, the mapping catheter 322 includes a selectively deployable stimulation electrode 324, which is shown in its deployed position, and a selectively deployable sense electrode 326, which is also shown in its deployed position. As will be appreciated from the below discussion of FIGS. 5A and 5B, which are used to show and describe an example implementation of the mapping catheter 322, actuators located on a handle of the mapping catheter 322 can be used to selectively deploy (aka expand) the various electrodes of the catheter. In FIG. 3B, by contrast, a selectively deployable stimulation electrode 334 is shown as being part of a distal portion of the guide catheter 302, instead of being part of the mapping catheter 322. The selectively deployable electrodes can be, for example, basket type electrodes, coiled wire electrodes, or laser cut electrodes, but are not limited thereto. In still other embodiments, a stimulation electrode that is located on a guidewire can be used to deliver the one or more electrical stimulation pulses.

The high level flow diagram of FIG. 4A is now used to describe methods, according to certain embodiments of the present technology, for use in evaluating neural activity of renal nerves in tissue surrounding a renal artery (e.g., 104) of a patient for which a renal denervation procedure has been or is going to be performed. Referring to FIG. 4A, step 402 involves inserting a distal portion of a guide catheter (e.g., 302) through the abdominal aorta (e.g., 106) of a patient such that the distal portion of the guide catheter is positioned adjacent to a renal artery ostium (e.g., 110) located along a lateral aspect of the abdominal aorta (e.g., as shown in FIG. 3A), or such that the distal portion of the guide catheter is inserted through the renal artery ostium and into a proximal portion of the renal artery of the patient (e.g., as shown in FIG. 3B). Step 404 involves using the guide catheter (e.g., 302) to insert a distal portion of a mapping catheter (e.g., 322) through a distal most end of the guide catheter such that the distal portion of the mapping catheter is positioned within the renal artery (e.g., 104) of the patient. Additional details of steps 402 and 404 can be appreciated from the above discussion FIGS. 3A and 3B.

While the distal portion of the mapping catheter (e.g., 322) is positioned within the renal artery (e.g., 104) of the patient, step 406 involves using a stimulation electrode (e.g., 324 or 334) positioned in the abdominal aorta (e.g., 106), preferably within millimeters (mm) of the renal artery ostium (e.g., 110), to deliver one or more electrical stimulation pulses to thereby evoke a neural response from at least some of the renal nerves in tissue surrounding the renal artery. Depending upon the specific implementation, such a stimulation electrode (e.g., 324) can be located on the mapping catheter (e.g., 322), e.g., as shown in FIG. 3A. Alternatively, such a stimulation electrode (e.g., 334) can be located on a distal portion of the guide catheter (e.g., 302), e.g., as shown in FIG. 3B. In still other embodiments, not specifically shown, a stimulation electrode can be located on a distal portion of a guidewire that is positioned within the abdominal aorta (e.g., 106). For an example, referring briefly back to FIG. 3A, the deployable electrode 324 can be used as the stimulation electrode, and the return electrode can be, e.g., a non-deployable ring electrode (not specifically shown in FIG. 3A) of the mapping catheter 322. For another example, referring briefly back to FIG. 3B, the deployable electrode 334 can be used as the stimulation electrode, and the return electrode can be, e.g., a non-deployable ring electrode (not specifically shown in FIG. 3B) of the guide catheter 302. Examples of non-deployable ring electrodes, which can be used as the return electrode, are described below with reference to FIGS. 5A and 5B. More specifically, electrode 525 in FIGS. 5A and 5B is an example of a non-deployable ring electrode that can be used as the return electrode.

The stimulation electrode can also be referred to as the stimulation cathode, while the return electrode can also be referred to as the stimulation anode. In certain embodiments, the electrical stimulation pulse(s) is/are delivered between a stimulation electrode (on the mapping catheter, the guide catheter, or a guidewire) and a return surface electrode (not specifically shown) that is positioned on skin of the patient. In certain embodiments, the stimulation electrode is a deployable stimulation electrode (e.g., 324 or 334) that is deployed such that an outer circumference of the deployable stimulation electrode is in contact with an inner wall of the abdominal aorta, preferably within 10 mm of the renal artery ostium (e.g., 110). It would also be possible that a stimulation electrode that is positioned within the abdominal aorta (e.g., 106) is located on a further catheter or wire that extends through a distal end of a guide catheter (e.g., 302) or through an opening along a length of the guide catheter.

Referring again to the flow diagram of FIG. 4A, step 408 involves (while the distal portion of the mapping catheter is positioned within the renal artery of the patient) using a sense electrode positioned in the renal artery, downstream from the stimulation electrode (e.g., 324), to sense the neural activity (and more specifically, renal nerve activity) evoked in response to the one or more electrical stimulation pulses. In certain embodiments, the sense electrode (e.g., 326) is located on the mapping catheter (e.g., 322). In certain embodiments, the sense electrode is a deployable sense electrode (e.g., 326) that is deployed such that an outer circumference of the deployable sense electrode is in contact with an inner wall of the renal artery, downstream of the deployable stimulation electrode by at least 1.5 cm, and preferably at by least 2.0 cm, and more preferably by at least 3.0 cm. It is believed that it is beneficial to maximize the distance between the stimulation electrode(s) and the sense electrode(s), because the further downstream the sense electrode(s) is/are from the stimulation electrode(s), the less chance that the sense electrode(s) will detect crosstalk or other noise generated by the electrical stimulation pulse(s) themselves, since the desire is to just sense the neural response and avoid sensing crosstalk and other potential noise. In certain embodiments, the deployable sense electrode (e.g., 326) is used together with another sense electrode (e.g., a non-deployable ring electrode or tip electrode) to sense the neural response. In such embodiments, the other sense electrode can also be located on the mapping catheter downstream of the stimulation electrode by at least 1.5 cm, and preferably at by least 2.0 cm, and more preferably by at least 3.0 cm. Examples of non-deployable ring electrodes, which can be used as sense electrodes, are described below with reference to FIGS. 5A and 5B. More specifically, electrode 527 in FIGS. 5A and 5B is an example of a non-deployable ring electrode that can be used as a sense electrode. It would also be possible that the deployable sense electrode is used together with another sense electrode to sense the neural response, wherein the other sense electrode (not specifically shown) is a surface electrode that is positioned on skin of the patient.

Step 410 involves using the sensed neural electrical activity, evoked in response to the electrical stimulation pulse(s) delivered at step 406, as a pre-denervation or post-denervation measurement associated with a renal denervation procedure. For example, the sensed evoked neural electrical activity can be used as baseline neural electrical activity, sensed prior to a renal denervation procedure, so that the baseline neural electrical activity can later be compared to evoked neural electrical activity sensed following a renal denervation procedure to determine the efficacy of the denervation procedure. Such a denervation procedure can be performed by using a transducer to emit ablation energy into a treatment region. Such ablation energy can be ultrasound energy, pulsed electric field RF ablation energy, or microwave ablative energy, but is not limited thereto. Alternatively, some other means of ablation may be used, such as chemical and/or cryotherapy. Prior to performing such a renal denervation procedure, pre-denervation neural activity (aka baseline neural activity) could be sensed using the same catheter that is used to perform the denervation procedure, or a separate catheter (e.g., a mapping catheter), so that the pre-denervation neural activity can be compared to post-denervation neural activity, for the purpose of determining whether the denervation procedure was successful, and thus, to determine whether the denervation procedure can be terminated, or whether addition ablation energy should be delivered. Further, even if pre-denervation neural activity (aka baseline neural activity) is not sensed, it can still be useful to sense post-denervation neural activity, for the purpose of determining whether the denervation procedure was successful, and thus, can be terminated, or whether addition ablation energy (or some other means of additional ablation) should be delivered.

In accordance with certain embodiments, step 410 involves determining, based on the sensed neural electrical activity (sensed at step 408), whether or not further ablation energy should be delivered from a location along a longitudinal length of the renal artery. For example, in accordance with certain embodiments of the present technology, a magnitude of the evoked neural electrical activity sensed at step 410 can be compared to a threshold, and if the magnitude of the evoked neural electrical energy is less than the threshold there can be a determination at step 410 that the renal denervation procedure was successful, and that no further renal denervation is needed. By contrast, if the magnitude of the evoked neural electrical response exceeds the threshold there can be a determination at step 410 that the renal denervation procedure was not successful, and that further renal denervation is still needed. If further denervation of the renal artery is still needed, then further ablation energy (or some other means of additional ablation) can be delivered. The ablation energy can be delivered, e.g., using an ultrasound transducer. As noted above, other types of ablation mechanisms can be used, such as, but not limited to microwave, cryotherapy, chemical, and/or pulsed electric field RF ablation.

As noted above, in certain embodiments the neural electrical activity that is sensed at step 408 can be an evoked neural electrical response to stimulation energy that is delivered using one or more electrodes of a catheter, which catheter may also include at least one of the sense electrodes used at step 408, and which catheter may or may not be the same catheter used to deliver the ablation energy, depending upon the specific implementation. The pair of electrodes that are used to deliver the electrical stimulation that evokes a neural electrical response can be referred to herein as first and second stimulation electrodes. In certain embodiments, both the first and the second stimulation electrodes are positioned in the abdominal aorta (e.g., 106), preferably within 10 mm of the of the renal artery ostium (e.g., 110). In other embodiments, only the first stimulation electrode is positioned in the abdominal aorta within 10 mm of the of the renal artery ostium, while the second stimulation electrode is located proximal (aka upstream of) the first stimulation electrode. One of the first and the second stimulation electrodes can be configured as the stimulation anode, while the other is configured as the stimulation cathode. In still other embodiments, the second stimulation electrode (that is used as the return electrode), rather than being located on the same catheter as the first stimulation electrode, is located on a distal end of an introducer sheath or guide catheter (e.g., 302) that is used to insert the catheter (e.g., 322) that includes the first stimulation electrode into the renal artery. In still another embodiment, the second stimulation electrode (that is used as the return electrode), rather than being located on the same catheter as the first stimulation electrode, is an external skin electrode. Other variations are also possible and within the scope of the embodiments described herein.

It is believed that there are certain potential benefits to delivering electrical stimulation energy (in the form of one or more electrical stimulation pulses) from one or more electrodes positioned within the abdominal aorta (e.g., 106), in close proximity to the renal artery ostium (e.g., 110) and the aorticorenal ganglia (e.g., 112), when one or more sensing electrodes downstream thereof and within the renal artery is/are used to sense the evoked neural electrical response to the stimulation energy. Accordingly, in certain embodiments, step 406 involves using a stimulation electrode positioned in the abdominal aorta (e.g., 106) to deliver one or more electrical stimulation pulses to an aorticorenal ganglia (e.g., 112) thereby evoking a neural electrical response from at least some of the renal nerves in tissue surrounding the renal artery (e.g., 104). The stimulation electrode can be located on the mapping catheter (e.g., 322), on the distal portion of the guide catheter (e.g., 302), or on a guidewire (not specifically shown). One potential benefit of such embodiments is that there is a higher probability of stimulating the aorticorenal ganglia (e.g., 112), which should recruit more renal nerve fibers to evoke a better neural electrical response than could be evoked if the stimulation energy was instead only delivered using one or more electrodes positioned within the renal artery itself, downstream of the renal artery ostium. In other words, it is believed that stimulating the aorticorenal ganglia (e.g., 112) using an electrode positioned within the abdominal aorta (e.g., 11) will result in stimulation of a larger percentage of the renal nerve fibers within tissue surrounding the renal artery, compared to if the stimulation is delivered from within the renal artery downstream of the renal artery ostium and downstream of the aorticorenal ganglia. For this reason, it is believed that embodiments of the present technology described herein can provide for a greater evoked neural electrical response that is more readily capable of being sensed using one or more sense electrodes of a catheter.

Another potential benefit of delivering electrical stimulation energy (in the form of one or more electrical stimulation pulses) from one or more electrodes within the abdominal aorta (e.g., 106) is that this allows for a greater spatial separation between the one or more stimulation electrodes (used to deliver the stimulation energy) and the one or more sense electrodes positioned within the renal artery (which one or more sense electrodes are used to sense the evoked neural electrical response). The greater the separation between the one or more stimulation electrodes (used to deliver the electrical stimulation energy) and the one or more sense electrodes (positioned within the renal artery) used to sense the evoked neural electrical response, the lower the chance that the evoked neural electrical response is corrupted by crosstalk or other noise caused by the generation of the electrical stimulation energy.

In the embodiments described above, the evoked neural electrical response that is sensed within a window of time following delivery of stimulation energy is capable of being sensed using one or more electrodes on a catheter inserted into the renal artery, without requiring that the catheter include a blood pressure sensor or other type of physiologic sensor, which would likely make the catheter larger, more complex, more expensive, and less reliable than desired. Further, it is believed that sensing for a physiologic response to delivery of electrical stimulation energy to renal nerves, such as sensing for a change in blood pressure in response to the delivery of stimulation energy, is less predictable and less reliable than sensing for an evoked neural electrical response to stimulation of renal nerves. Accordingly, it is believed that sensing for an evoked neural electrical response to stimulation energy delivered to renal nerves provides for many improvements over sensing for a physiologic response when there is a desire to quantify renal nerve function prior to a renal denervation procedure and/or following a renal denervation procedure.

Since the inner diameter of an abdominal aorta (e.g., 110) is typically larger than the inner diameter of a renal artery (e.g., 104) extending from the abdominal aorta, a larger deployable stimulation electrode (e.g., a basket, coiled wire, or laser cut stimulation electrode) can likely be deployed within the abdominal aorta than can be deployed within the renal artery. This should allow for a greater surface area of the deployed stimulation electrode to be placed in contact with an inner wall of the abdominal aorta than could be placed in contact with an inner wall of the renal artery, which could potentially cause more renal nerves to be stimulated, and thereby could provide for a greater evoked neural electrical response.

Additionally, if there is stenosis (i.e., narrowing) of a renal artery due to disease, the stenosis often occurs near the renal artery ostium (e.g., 110), which may make if difficult and potentially dangerous to position and deploy a stimulation electrode within the renal artery, close to the renal artery ostium. Using certain embodiments of the present technology described herein, by instead positioning and deploying a stimulation electrode from within the abdominal aorta (e.g., 106), the aforementioned difficulty and potential danger could be avoided.

In accordance with certain embodiments, rather than just delivering the stimulation energy (in the form of one or more electrical stimulation pulses) from one or more electrodes positioned within the abdominal aorta, in close proximity to the renal artery ostium and the aorticorenal ganglia, additional stimulation energy (in the form of one or more additional electrical stimulation pulses) can also be delivered from one or more further stimulation electrode within a proximal portion of the renal artery, which could even further increase the percentage of renal nerves that are stimulated and thereby potentially evoke an even better neural response. The additional stimulation energy (delivered using electrode(s) within the renal artery) can be delivered simultaneously with the stimulation energy delivered using electrode(s) within the abdominal aorta. Alternatively, the delivery of the additional stimulation energy (delivered using electrode(s) within the renal artery) can be staggered in time according to the expected velocity of nerve signal propagation such that the renal nerve signals arrive at the same time at the sensing electrode downstream of the stimulation electrodes. More specifically, this can involve stimulating from within the aorta, waiting a brief delay, and then stimulating from within the renal artery, and then sensing an evoke response to both stimulations at the same time using the sensing electrode(s) within a distal portion of the renal artery. In certain such embodiments, parameters of the first stimulation energy can be taken into account when calculating the parameters for the second stimulation energy, so that overstimulation from within the renal artery is avoided.

One or more of the above described evoked neural electrical responses to stimulation energy being delivered, which is/are sensed from one or more sense electrodes within the renal artery, can be a direct neural response of renal nerves that are located within tissue surrounding the renal artery. It is also possible that if the stimulation energy is of a great enough amplitude and delivered for a sufficiently long duration, that a systematic response from the brain may also be caused and sensed by the one or more sense electrodes within the renal artery. It is believed that such a systematic response, if it were to occur, would occur a short delay after the direct neural response.

Reference is now made to FIG. 4B, which is used to describe a specific implementation of the methods introduced above with reference to FIG. 4A, for use in evaluating neural activity of renal nerves in tissue surrounding a renal artery (e.g., 104) of a patient for which a renal denervation procedure has been or is going to be performed. Steps 402, 404, 408, and 410 in FIG. 4B are the same as in FIG. 4A, and thus are labeled the same, and need not be described again in detail, because such steps were already described above with reference to FIG. 4A. Referring to FIG. 4B, step 406′ involves using a stimulation electrode positioned in the abdominal aorta to deliver one or more electrical stimulation pulses to an aorticorenal ganglia, to thereby evoke a neural electrical response from at least some of the renal nerves in tissue surrounding the renal artery. In certain such embodiments, the stimulation electrode can be located on the mapping catheter, on the distal portion of the guide catheter, or on a guidewire.

One potential challenge to effectively stimulating the aortorenal ganglia is the relatively large distance (e.g., approximately 10 mm or more) between the aortorenal ganglia and aorta wall or the abdominal aorta. However, this challenge can be addressed and overcome by using appropriate electrical stimulation parameters and an appropriate stimulation electrode. With electric stimulation, the current drops quickly with the distance from the electrode. In order to increase the probability that the aortorenal ganglia is effectively stimulated, a relatively large electrode and/or a relatively high stimulation current can be selected and used to provide for an increased penetration depth that is sufficient to stimulate the aortorenal ganglia. Alternatively, or additionally, anodic stimulation can be utilized, which is believed to deliver more precise activation of nerves at a distance. Such an approach combined with proper electrode geometry, pulse amplitude, and pulse duration can be used to effectively stimulate the aortorenal ganglia, which as noted above, are likely about 10 mm or more away from the aortic wall. Alternatively, an ultrasound thermal effect can be used to stimulate the aortorenal ganglia, by using ultrasound to heat tissue surrounding the aortorenal ganglia to a temperature within the range of approximately 39 degrees to 48 degrees Celsius to activate a neural response from the aortorenal ganglia. More specifically, tissue can be heated to a temperature within the range of approximately 39 degrees to 48 degrees Celsius by emitting defocused or focused ultrasound energy within a frequency range of approximately 5 MHz to 15 MHz.

In certain embodiments, it can be assumed that the aortorenal ganglia is within about 10 mm of the renal artery ostium. Alternatively, imaging may be used to visualize and locate the position of the aortorenal ganglia. For example, ultrasound imaging using ultrasound frequencies above 5 MHz, and preferably at 10 MHz or above, can be used to reliably visualize the aortorenal ganglia. The aortorenal ganglion is relatively small, typically having a length within the range of approximately 1 mm to 2 mm. Accordingly, it would be preferable to utilize imaging that has a resolution of approximately 0.5 mm or smaller. Alternatively, Magnetic resonance imaging (MRI) can be used to visualize and locate the position of the aortorenal ganglia. Other types of imaging may be used while being within the scope of the embodiments described herein.

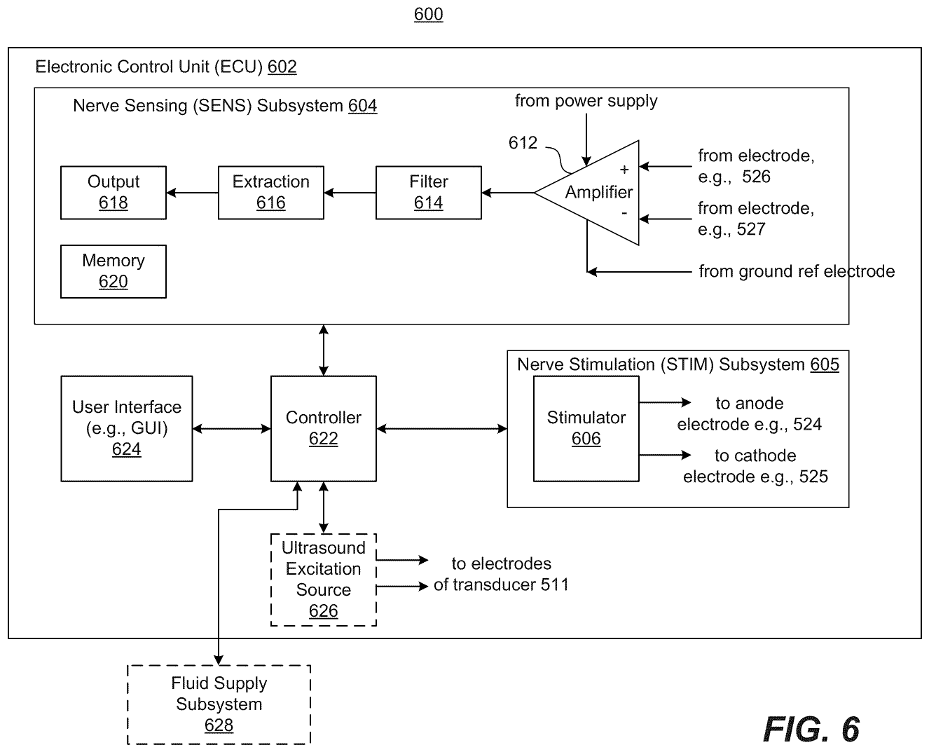

In certain embodiments, step 410 (of FIGS. 4A and 4B) can include various sub-steps. For example, step 410 can include determining one or more characteristics of the sensed neural activity of renal nerves, wherein the characteristic(s) is/are indicative of one or more of the size, function or health of the renal nerves for which neural activity is sensed and/or indicative of proximity of the renal nerves relative to the sensing electrode(s) of the catheter, but not limited thereto. In certain embodiments, such sub-steps can be performed by a controller 622 of a system 600 described below with reference to FIG. 6. Another sub-step can involve selecting one or more denervation parameters based on the characteristic(s) of the sensed neural activity of renal nerves, wherein the denervation parameter(s) is/are for use in performing a renal denervation procedure intended to denervate at least some of the renal nerves for which the neural activity was sensed. In accordance with certain embodiments, the selecting of renal denervation parameter(s) is performed using one or more processors, e.g., of an electrical control unit (ECU), an example of which is described below with reference to FIG. 6. Such an ECU can include a controller (e.g., 622 in FIG. 6), wherein the controller includes one or more processors. In certain embodiments, one or more renal denervation parameters is/are selected using one or more tables stored in memory (e.g., 620 in FIG. 6) of an ECU (e.g., 602 in FIG. 6) and accessed by at least one processor (e.g., of a controller 622 in FIG. 6). In other embodiments, one or more renal denervation parameters is/are selected using a machine learning model implemented by at least one of the one or more processors, or more generally, using artificial intelligence.

Another sub-step of step 410 may involve performing a renal denervation procedure using the selected renal denervation parameter(s) to thereby denervate at least some of the renal nerves for which the neural activity was sensed. This sub-step can involve a controller (e.g., 622) controlling a signal generator (e.g., 606) to generate signals for performing the renal denervation procedure using the selected one or more renal denervation parameters. The same mapping catheter 322 used at or to perform steps 404, 406/406′, and 408 is preferably also used to perform a renal denervation procedure using the selected renal denervation parameter(s) so as to eliminate the need to perform swapping of catheters and thereby simplify and reduce the time required to perform the renal denervation procedure. An example of a catheter that can be used at or to perform steps 404, 406/406′, and 408, as well as to perform a renal denervation procedure, is described below with reference to FIGS. 5A and 5B. However, it would also be possible to use a different catheter at or to perform steps 404, 406/406′, and 408, as well as to perform a renal denervation procedure. Further, it is also noted that it would also be possible to use one catheter to perform steps 404, 406/406′, and 408, and a different catheter to perform a renal denervation procedure.

In certain embodiments, characteristics of the evoked neural response of renal nerves sensed at step 408 can be quantified and used to tailor patient specific parameters of renal nerve destruction that is to be performed as part of a medical procedure. Such characteristics can include or be based on amplitudes of the multiple peaks and/or based on temporal spacings between multiple peaks of the sensed signal indicative of the evoked neural response of renal nerves. For example, an average amplitude of the multiple peaks can be determined and one or more renal denervation parameters can be selected (e.g., by the controller 622) based on the average amplitude of the multiple peaks. Alternatively, or additionally, a median amplitude of the multiple peaks can be determined and renal denervation parameters can be selected based on the median amplitude of the multiple peaks. Alternatively, or additionally, a curve can be fit to a portion of the signal indicative of the neural activity of renal nerves, and renal denervation parameters can be selected based on the area under the curve. Additionally details of how denervation parameters can be selected are described in commonly assigned U.S. patent application Ser. No. 18/182,821, titled USING CHARACTERISTICS OF NATIVE OR EVOKED SENSED NEURAL ACTIVITY TO SELECT DENERVATION PARAMETERS, filed Mar. 13, 2023, which is incorporated herein by reference, and some details of which are provided below.

In certain embodiments, characteristic(s) of the sensed neural activity of renal nerves is/are determined based on amplitudes of the multiple peaks and/or based on temporal spacings between the multiple peaks of the sensed signal indicative of the neural activity of renal nerves. For example, an average amplitude of the multiple peaks can be determined and one or more renal denervation parameters can be selected (e.g., by the controller 622) based on the average amplitude of the multiple peaks. Where the average amplitude of the multiple peaks is relatively large, e.g., above a specified threshold (e.g., of 2.0 μV), that means there is relatively high renal nerve activity that is sensed and that a relatively high amplitude and/or relatively long duration renal denervation therapy should be selected and used to perform the renal denervation procedure. Where the average amplitude of the multiple peaks is relatively small, e.g., below a specified threshold (e.g., of 2.0 μV), that means there is relatively low renal nerve activity that is sensed and that a relatively low amplitude and/or relatively short duration renal denervation therapy should be selected (e.g., by the controller 622). For a more specific example, once the average amplitude of the multiple peaks is below a specified threshold (e.g., of 2.0 μV), the amplitude and/or duration of the renal denervation therapy can be decreased by 50% compared to when the median amplitude of the multiple peaks is above the specified threshold.

Alternatively, or additionally, a median amplitude of the multiple peaks can be determined and renal denervation parameters can be selected based on the median amplitude of the multiple peaks. For example, a median amplitude of the multiple peaks can be determined and one or more renal denervation parameters can be selected (e.g., by the controller 622) based on the average amplitude of the multiple peaks. Where the median amplitude of the multiple peaks is relatively large, e.g., above a specified threshold (e.g., of 2.0 μV), that means there is relatively high renal nerve activity that is sensed and that a relatively high amplitude and/or relatively long duration renal denervation therapy should be selected and used. Where the median amplitude of the multiple peaks is relatively small, e.g., below a specified threshold (e.g., of 2.0 μV), that means there is relatively low renal nerve activity that is sensed and that a relatively low amplitude and/or relatively short duration renal denervation therapy should be selected (e.g., by the controller 622) and used. For a more specific example, once the median amplitude of the multiple peaks is below a specified threshold (e.g., of 2.0 μV), the amplitude and/or duration of the renal denervation therapy can be decreased by 50% compared to when the median amplitude of the multiple peaks is above the specified threshold.

Alternatively, or additionally, determining one or more characteristics of the sensed renal nerve activity (e.g., by the controller 622) can involve fitting a curve to a portion of the signal indicative of the neural activity of renal nerves, and determining an area under the curve, and renal denervation parameters can be selected based on the area under the curve. Where the area under the curve is relatively large, e.g., above a specified threshold, that means there is relatively high renal nerve activity that is sensed and that a relatively high amplitude and/or relatively long duration renal denervation therapy should be selected (e.g., by the controller 622) and used. Where the area under the curve is relatively small, e.g., below a specified threshold, that means there is relatively low renal nerve activity that is sensed and that a relatively low amplitude and/or relatively short duration renal denervation therapy should be selected (e.g., by the controller 622) and used. For a more specific example, once the area under the curve is below a specified threshold, the amplitude and/or duration of the renal denervation therapy can be decreased by 50% compared to when the area under the curve is above the specified threshold.

In each of the above embodiments, the renal denervation procedure can be considered complete when the average, median, or area under the curve is below a corresponding specified threshold. For example, in certain embodiments, where the average or median amplitude of multiple peaks is below 0.5 μV, then it can be concluded the neural activity of renal nerves is sufficient low such that no (or no further) renal denervation therapy is needed.

Each renal nerve that surrounds a renal artery will typically fire only once per cardiac cycle. Accordingly, where the temporal spacings between sensed peaks is small relative to a cardiac cycle length, that can be interpreted as there being many renal nerves firing during the cardiac cycle. By contrast, where the temporal spacing between sensed peak is large relative to a cardiac cycle length, that can be interpreted as there being relatively few renal nerves firing during the cardiac cycle. Such observations can be used to determine whether a renal denervation procedure, or a further renal denervation procedure, should be performed, and/or to select one or more renal denervation parameters.

When renal nerve fibers are ablated during a renal denervation procedure, such that the health of the renal nerve fibers has been diminished or the renal nerves have been destroyed, the amplitude and shape of a sensed signal indicative of the neural activity of renal nerves, its latency, and its synchrony will change, compared to such characteristics prior to the renal denervation procedure. In certain embodiments, one or more of these various characteristics, such as amplitude, shape, latency, and synchrony, are used to quantify the health of renal nerves.

In accordance with certain embodiments, convolution is used to determine how far renal nerve fibers are from a sensing electrode (e.g., 326, 327), or more generally from a sensing site (which can also be referred to as a recording site). Typically, the larger the amplitude of the sensed neural activity the more likely renal nerve fibers are close to the sensing site, and the smaller the amplitude of the sensed neural activity the more likely the renal nerve fibers are far from the sensing site. Assume for example the renal nerve activity of two different renal nerve fibers are sensed using a sensing electrode on a catheter, and that one of the renal nerve fibers is relatively far away from the sensing site, while another one of the renal nerve fibers is relatively close to the sensing site. Also assume that both the relatively far and the relatively close renal nerve fibers fire at the same time in response to stimulation energy that is intended to evoke a neural response of renal nerves. When a catheter is used to sense the above described neural activity of renal nerves, characteristics of both the relatively far and the relatively near renal nerve fibers can be distinguished from one another in a sensed signal, which can enable a system and/or physician to approximate how far the different renal nerve fibers are from the sensing site, as well as the size of such renal nerve fibers, which are examples of characteristics of sensed neural activity of renal nerves that can be determined (e.g., by the controller 622).

In certain embodiments, sensed neural activity of renal nerves following a renal denervation procedure is compared to baseline neural activity of renal nerves sensed prior to the denervation procedure to determine the efficacy of the renal denervation procedure. Based on such a comparison, there can be a determination of whether the renal denervation procedure was sufficiently successful such that it could be terminated, or whether further ablation energy should be applied because sufficient renal nerve destruction has not yet been achieved. After enough data has been collected from a patient population, it may also be possible to analyze neural activity of renal nerves from a patient without requiring any baseline measurements. However, until such sufficient patient population data is collected, determining baseline measurements prior to a procedure, and comparing those to post-procedure measurements, is likely a good way to quantify the efficacy of the renal denervation procedure and to determine whether additional renal denervation treatment is needed.

In certain embodiments, neural activity of renal nerves can be sensed (e.g., by the sensing circuit 604) and used (e.g., by the controller 622) to diagnose a disease state of a patient, such as to diagnose a patient as having hypertension, since a patient with hypertension will have a renal nerve activity signature indicative of hypertension that is distinguishable from the renal nerve activity signature of a healthy patient that is not experiencing hypertension.

Embodiments of the present technology can be implemented using various different catheter implementations, and thus, are not limited to use with any specific catheter and or system of which a catheter is a part. Nevertheless, for completeness, an example catheter and system that can be used to implement embodiments of the present technology are described below. More specifically, FIGS. 5A, 5B and 6 are used to describe an example mapping catheter and system that can be used to implement certain embodiments of the present technology that were described above. Such a system can also be referred to as an apparatus herein.

### Example Catheter

FIG. 5A shows a catheter 502 with its selectively deployable electrodes 524 and 526 in their non-deployed positions, wherein the catheter 502 can be used as the mapping catheter 322 discussed above. The catheter 502 includes a catheter handle 512 and a catheter shaft 522. In addition to including the selectively deployable electrodes 524 and 526, the catheter shaft 522 is also shown as including a non-deployable electrode 525 that is proximal to the selectively deployable electrode 524, and a non-deployable electrode 527 that is distal the selectively deployable electrode 526. The selectively deployable electrode 524 can also be referred to as the proximal selectively deployable electrode 524, or more succinctly as the proximal electrode 524, or even more succinctly as the electrode 524. The selectively deployable electrode 526 can also be referred to as the distal selectively deployable electrode 526, or more succinctly as the distal electrode 526, or even more succinctly as the electrode 526. The catheter shaft 522 can also be referred to more succinctly herein as the shaft 522. The catheter 502 can be a specific implementation of the catheter 322 shown in and discussed above with reference to FIG. 3. Referring briefly back to FIG. 3A, the proximal selectively deployable electrode 524 can be used as the stimulation electrode 324, and the distal selectively deployable electrode 526 can be used as the sense electrode 326.

Referring again to FIG. 5A, the catheter 502 is also shown as including an optional balloon 513 positioned longitudinally between the electrodes 524 and 526, wherein the optional balloon 513 is selectively inflatable and deflatable. The balloon 513 can also be referred as a selectively inflatable balloon 513, a selectively deployable balloon 513, or more succinctly as a balloon 513. When the balloon 513 is deflated, it can also be referred to as being non-inflated or in its non-deployed position. When the balloon 513 is inflated, it can also be referred to as being in its deployed position. As will be described in additional detail below, the balloon 513 can be selectively inflated by injecting a fluid into the balloon 513, and the balloon 513 can be selectively deflated by removing the fluid from the balloon 513. The balloon 513 can be made of an electrically insulating material such as polyamide, polyethylene terephthalate, or thermoplastic elastomer. In specific embodiments the balloon 513 is made from nylon, a polyimide film, a thermoplastic elastomer (such as those marked under the trademark PEBAX™), a medical-grade thermoplastic polyurethane elastomers (such as those marketed under the trademark PELLETHANE™), pellethane, isothane, or other suitable polymers or any combination thereof, but is not limited thereto.

The catheter handle 512, which can also be referred to more succinctly as the handle 512, includes actuators 514, 516, and 518, which can be used to selectively deploy the electrodes 524, 526, as well as to adjust a longitudinal distance between the electrodes 524, 526, as will be described in additional detail below. The actuators 514, 516, and 518 are respectively slidable within slots 515, 517, and 519 in the handle 512, and thus, the actuators 514, 516, and 518 can also be referred to as sliders. The catheter handle 512 is also shown as including a fluidic inlet port 534a and a fluidic outlet port 534b.

A fluid (e.g., expelled from a pressure syringe) can enter a fluid lumen (in the catheter shaft 522), via the fluidic inlet port 534a of the catheter 502, and then enter and at least partially fill the balloon 513. Fluid can be drawn from the balloon 513 (e.g., using a vacuum syringe) through another fluid lumen (in the catheter shaft 522) and out the fluidic outlet port 534b of the catheter 502. In this manner, the fluid can be used to selectively inflate and selectively deflate the balloon 513. In certain embodiments, fluid can be simultaneously injected into and removed from the balloon 513 to thereby circulate the fluid through the balloon 513.

The catheter 502 can also be referred to as an intraluminal microneurography probe 502, or more succinctly, as a probe 502. A cable 504, which extends from a proximal portion of the handle 512, provides for electrical connections between the catheter 502 (and more specifically, the electrodes thereof) and an electrical control unit (ECU), an example of which is described below with reference to FIG. 6.

Still referring to FIG. 5A, an optional transducer 511 is shown as being within a balloon 513. The transducer 511 is an example of an ablation element that is included on the shaft 522 and configured to ablate nerve tissue using ultrasound energy. In other embodiments, the transducer and balloon may be replaced by a helical structure carrying a plurality of electrodes configured to deliver RF and/or pulsed electric field RF energy. In other embodiments, the transducer and balloon may be replaced by a microwave transmitting element within an expandable centering element. In other embodiments, the transducer may be replaced by a cryotherapeutic applicator. In other embodiments, the transducer and balloon may be replaced by an infusion needle configured to deliver an ablative chemical to the renal nerves.

Where a transducer 511 is within the balloon 513, the fluid that is circulated through the balloon 513 can be referred to as a cooling fluid that is used to cool the transducer 511 and/or to cool a portion of a biological lumen (e.g., renal artery) that the balloon 513 is within, and/or to cool the biological tissue surrounding the lumen. It is also possible that the catheter 502 is devoid of the transducer 511 or other ablative means, and that a separate catheter that includes a transducer or other ablative means is used to deliver ablation energy. Where the catheter 502 is devoid of the transducer 511 or other ablative means, one or more electrodes of the catheter 502 can be used for sensing native neural activity. One or more electrodes of the catheter 502 can be used for delivering stimulation energy and one or more further electrodes of the catheter 502 can be used for sensing an evoked neural electrical response to the stimulation energy.

When the catheter 502 is inserted into a biological lumen, such as a renal artery, it is the distal portion of the catheter 502 (and more specifically the shaft 522) that is inserted into the biological lumen, and the proximal end of the catheter 502 (and more specifically the handle 512) that is used to maneuver the catheter 502. In the embodiment shown in FIGS. 5A and 5B, the electrode 526 can also be referred to as a distal selectively deployable electrode 526 as noted above, since it located closer to the distal end of the catheter 502 than to the proximal end of the catheter 502; and the electrode 524 can also be referred to as a proximal selectively deployable electrode 524 as noted above, since it is located closer to the proximal end of the catheter 502 than to the distal end of the catheter 502. For similar reasons, the electrode 525 can be referred to as the proximal non-deployable electrode 525, and the electrode 527 can be referred to as the distal non-deployable electrode 527.

FIG. 5B shows the catheter 502 with the electrodes 524 and 526 in their deployed (aka expanded) positions. In certain embodiments, the proximal selectively deployable electrode 524 is configured to be deployed (aka expanded) in response to the actuator 514 being slid in the proximal direction indicated by the arrow 544 in FIG. 5B. In such an embodiment, the proximal electrode 524 can be returned to its non-deployed (aka non-expanded or retracted) position in response to the actuator 514 being slid in the distal direction opposite the arrow 544 in FIG. 5B. More generally, the actuator 514 is used to selectively expand and retract the electrode 524. Referring briefly back to FIG. 3A, in certain embodiments, where the proximal selectively deployable electrode 524 provides the stimulation electrode 324, the proximal selectively deployable electrode 524 can be deployed while located within the abdominal aorta, and the distal selectively deployable electrode 526 can provide the sense electrode 326 and be deployed while located within the renal artery.

Referring again to FIG. 5B, in accordance with certain embodiments, the longitudinal distance between the distal electrode 526 and the proximal electrode 524 can be reduced by sliding the actuator 518 in the proximal direction indicated by the arrow 548 in FIG. 5B. Thereafter, the longitudinal distance between the distal electrode 526 and the proximal electrode 524 can be increased, if desired, by sliding the actuator 518 in the distal direction opposite the arrow 548 in FIG. 5B. More generally, the actuator 518 is used to adjust the longitudinal distance between the electrodes 524 and 526. The longitudinal distance between the proximal and distal electrodes 524, 526 can be any distance between the maximum and minimum longitudinal distance as controlled by a user using the actuator 518. In accordance with certain embodiments, the electrode 524 is configured to be deployed in response to the actuator 514 being slid in the proximal direction indicated by the arrow 544 in FIG. 5B. In accordance with certain embodiments, the distal electrode 526 is configured to be deployed in response to the actuator 516 being slid in the proximal direction indicated by the arrow 546 in FIG. 5B. In such an embodiment, the distal electrode 526 can be returned to its non-deployed position in response to the actuator 516 being slid in the distal direction opposite the arrow 546 in FIG. 5B. More generally, the actuator 516 is used to selectively expand and retract the electrode 526. Other variations are also possible and within the scope of the embodiment described herein.

Each of the selectively deployable electrodes 524, 526 can be made, for example, of a unitary nitinol tube that is laser cut to include apertures or openings having a predetermine pattern. In FIGS. 5A and 5B, each of the electrodes 524, 526 has laser cut spiral apertures that extend between proximal and distal portions of each of the electrodes 524, 526. The spiral apertures in each of the electrodes 524, 526 enable each of the electrodes to be selectively transitioned between their non-deployed and deployed positions. The apertures that are cut into the electrodes 524, 526 can have other shapes besides being spiral, so long as the apertures enable the electrodes to be transitioned between non-deployed and deployed positions. The selectively deployable electrodes 524, 526 can alternatively be mesh electrodes or spiral electrodes made of one or more electrically conductive wires, optionally with portions thereof being insulated. Other variations are also possible and within the scope of the embodiments described herein.

The catheter 502 can be configured to be introduced into a biological lumen, such as an artery, in a location near a body organ, such as a kidney. The catheter 502 can be introduced via a guide catheter (e.g., 302) that is advanced to the intended catheter location in the biological lumen, and then withdrawn sufficiently to expose the shaft 522 to the biological lumen (e.g., renal artery). Once the shaft 522 is within the biological lumen, one of the electrodes 524, 526 can be deployed (aka expanded) using one of the actuators 514, 516 such that it is in contact with a portion of a circumferential interior wall of the biological lumen. The longitudinal distance between the electrodes 524 and 526 can then be adjusted, if desired, using the actuator 518. The other one of the electrodes 524, 526 can then be deployed (aka expanded) such that it is in contact with another portion of the circumferential interior wall of the biological lumen.

For example, where the catheter is inserted into a renal artery close to a kidney, the electrodes can be positioned near a nerve bundle that connects the kidney to the central nervous system, as the nerve bundle tends to approximately follow the artery leading to most body organs. The nerve bundle tends to follow the artery more closely at the end of the artery closer to the kidney, while spreading somewhat as the artery expands away from the kidney. As a result, it is desired in some examples that the catheter shaft 522 is small enough to introduce relatively near the kidney or other organ, as nerve proximity to the artery is likely to be higher nearer the organ.

Once the catheter 502 is in place, a practitioner can use instrumentation (e.g., the ECU 602) coupled to the electrodes to stimulate one or more nerves, and monitor for evoked nerve response signals used to characterize the nervous system response to certain stimulus. The transducer 511 and/or other ablative means are configured to ablate nerve tissue, such as by using ultrasound, RF, pulsed electric field RF, microwave, cryotherapy, or other energy or chemical means. Additionally, the catheter 502 can actively stimulate one or more nerves and sense resulting neural signals in between applications of ablation energy via the transducer 511, enabling more accurate control of the degree and effects of nerve ablation. In other examples, a catheter 502 lacking a transducer or other ablative means can be removed via a sheath, and an ablation probe (aka catheter) inserted, with the ablation probe removed and the catheter 502 reinserted to verify and characterize the effects of the ablation probe.

Any one or more electrodes of the catheter 502 can be selectively used to deliver stimulation energy to nerves surrounding a biological lumen. Similarly, any one or more electrodes of the catheter can be selectively used to sense neural activity of nerves surrounding a biological lumen, which can be spontaneous neural activity, or evoked neural activity.

For much of the below discussion, it is assumed that the transducer 511 is an ultrasound transducer that can be activated to deliver unfocused ultrasonic energy radially outwardly so as to suitably heat, and thus treat, tissue within the target anatomical region. The transducer 511 can be activated at a frequency, duration, and energy level suitable for treating the targeted tissue. In one nonlimiting example, the unfocused ultrasonic energy generated by the transducer 511 may target select nerve tissue of the subject, and may heat such tissue in such a manner as to neuromodulate (e.g., fully or partially ablate, necrose, or stimulate) the nerve tissue.

In accordance with certain embodiments, the transducer 511 includes a piezoelectric transducer body that comprises a hollow tube of piezoelectric material having an inner surface and an outer surface, with an inner electrode disposed on the inner surface of the hollow tube of piezoelectric material, and an outer electrode disposed on the outer surface of the hollow tube of piezoelectric material. In such embodiments, the hollow tube of piezoelectric material is an example of the piezoelectric transducer body. The hollow tube of piezoelectric material, or more generally the piezoelectric transducer body, can be cylindrically shaped and have a circular radial cross-section. However, in alternative embodiments the hollow tube of piezoelectric material can have other shapes besides being cylindrical with a circular radial cross-section. Other cross-sectional shapes for the hollow tube of piezoelectric material, and more generally the piezoelectric transducer body, include, but are not limited to, an oval or elliptical cross-section, a square or rectangular cross-section, pentagonal cross-section, a hexagonal cross-section, a heptagonal cross-section, an octagonal cross-section, and/or the like. The hollow tube of piezoelectric material, and more generally the piezoelectric transducer body, can be made from various different types of piezoelectric material, such as, but not limited to, lead zirconate titanate (PZT), polyvinylidene fluoride (PVDF), or other presently available or future developed piezoelectric ceramic materials. In other embodiments, the transducer 511 can be made of other materials and/or can have other shapes.

In certain embodiments, the transducer 511 is an ultrasound transducer configured to deliver acoustic energy in the frequency range of 8.5 to 9.5 MHz. In certain embodiments, the transducer is configured to deliver acoustic energy in the frequency range of 8.7-9.3 MHz or 8.695-9.304 MHz. Transducers delivering acoustic energy in the frequency range of 8.7-9.3 MHz have been shown to produce ablation up to mean depths of 6 mm. The piezoelectric transducer body is configured to generate ultrasonic waves in response to a voltage being applied between the inner and outer electrodes. One or both of the inner and outer electrodes can be covered by an electrical insulator to inhibit (and preferably prevent) a short circuit from occurring between the inner and outer electrodes when the ultrasound transducer is placed within an electrically conductive fluid and a voltage is applied between the inner and outer electrodes. Such an electrical insulator can be parylene, and more specifically, a parylene conformal coating, but is not limited thereto. An excitation source (e.g., 626 in FIG. 6) may be electrically coupled to inner and outer electrodes of the transducer 511, and may actuate the transducer 511 by applying a voltage between the inner and outer electrodes (or any other pair of electrodes), so as to cause the piezoelectric material of the piezoelectric transducer body to generate an unfocused ultrasonic wave that radiates radially outwardly.

### Example Electrical Control Unit (ECU)

FIG. 6 is a high level block diagram of an electrical control unit (ECU) 602 that is configured to be in electrical communication with a catheter, such as the catheter 502 described above. The ECU 602, and the catheter (e.g., 502) to which the ECU 602 is electrically coupled via a cable (e.g., 504), can be referred to more generally as a system 600. The ECU 602 can process a received signal to produce an output signal, and present information including information about the output signal, the received signal, or processing information. Such a system can be used, for example, in diagnostic procedures for assessing the status of a patient's nervous activity proximate a biological lumen, such as a vein or an artery, e.g., a renal artery, or another type of blood vessel. Such a system can be additionally, or alternatively, be used to select preferred denervation parameters for use in a denervation procedure. As will be described in additional details below, a same catheter that is used to assess the status of a patient's nervous activity proximate and/or select preferred denervation parameters can also be used to perform a denervation procedure. Alternatively, it is possible that the catheter that is used to perform a denervation procedure differs from the catheter that is used to assess that status of a patient's nervous activity proximate a biological lumen, in which case different catheters can be swapped in and out of a biological lumen during a procedure.

Still referring to FIG. 6, the ECU 602 includes a stimulator 606 electrically coupled to a selected pair of electrodes (e.g., 524 and 525) of the catheter 502. The stimulator 606 which is part of a STIM circuit or subsystem 605, can selectively emit electrical signals (including electrical stimulation pulses) having a specific voltage, amperage, duration, duty cycle and/or frequency of application that will cause nerve cell activation. For an example, the electrode 527 can be connected as the stimulation anode and the electrode 526 can be connected as the stimulation cathode, or vice versa. For another example, the electrode 524 can be connected as the stimulation anode and the electrode 525 can be connected as the stimulation cathode, or vice versa. Switches, which are not specifically shown, can be used to selectively control how various electrodes (e.g., 524, 525, 526 and 527) are coupled to various nodes of the ECU 602, such as to the input terminals of the amplifier 612, or to the output terminals of the stimulator 606.

Upon receiving the stimulation signal produced by the stimulator 606, the electrodes of the catheter 502 that are connected as stimulation electrodes (e.g., 524 and 525) can apply electrical energy to a patient's nerves through the biological lumen wall based on the received signal. Such stimulus can have any of a variety of known waveforms, such as a sinusoid, a square wave form or a triangular wave form, but is not limited thereto. In various examples, the stimulation can be applied for durations between approximately 0.05 milliseconds (msec) and approximately 8 msec. It is noted that the term approximately as used herein to specify a value means+/−10% of the value, e.g., approximately 8 msec means 8 msec+/−10%, which is 8 msec+/−0.8 msec.

The stimulation of nerves can be performed to evoke an elicited potential, which can cause such a potential to propagate in every direction along the nerve fibers. More generally, the STIM subsystem 605 can be used to deliver electrical stimulation via a selected pair of electrodes in order to evoke a neural response, and the SENS subsystem 604 can be used to sense the evoked neural electrical response.

In some embodiments, the ECU 602 can digitally sample the signal sensed using a pair of electrodes to receive the electrical signal from the catheter 502. In alternate embodiments, the signal can be recorded as an analog signal. When receiving an electrical signal from the electrodes on the catheter 502, the ECU 602 can perform filtering and/or other processing steps on the signal. Generally, such steps can be performed to discriminate the signal of interest sensed by the catheter from any background noise within the patient's vasculature such that the resulting output is predominantly the signal from nerve cell activation. In some instances, the ECU 602 can modulate the electrical impedance of the signal receiving portion in order to accommodate the electrical properties and spatial separation of the electrodes mounted on the catheter in a manner to achieve the highest fidelity, selectively and resolution for the signal received. For example, electrode size, separation, and conductivity properties can impact the field strength at the electrode/tissue interface.

Additionally or alternatively, the ECU 602 can comprise a headstage and/or an amplifier to perform any of offsetting, filtering, and/or amplifying the signal received from the catheter. In some examples, a headstage applies a DC offset to the signal and performs a filtering step. In some such systems, the filtering can comprise applying notch and/or band-pass filters to suppress particular undesired signals having a particular frequency content or to let pass desired signals having a particular frequency content. An amplifier can be used to amplify the entire signal uniformly or can be used to amplify certain portions of the signal more than others. For example, in some configurations, the amplifier can be configured to provide an adjustable capacitance of the recording electrode, changing the frequency dependence of signal pick-up and amplification. In some embodiments, properties of the amplifier, such as capacitance, can be adjusted to change amplification properties, such as the resonant frequency, of the amplifier.

In the illustrated embodiment of FIG. 6, the ECU 602 includes an amplifier 612 including a non-inverting (+) input terminal, an inverting (−) input terminal, a power supply input terminal, and a ground or reference terminal. As can be appreciated from FIG. 6, the non-inverting (+) input terminal can be coupled to the electrode 526, the inverting (−) input terminal can be electrically coupled to the electrode 527, the power supply input terminal is electrically coupled to a voltage source (e.g., a reference voltage generator), and the ground or reference terminal is electrically coupled to a ground reference electrode, which can be located on the catheter 502, can be located on a distal end of an introducer sheath, or can be located on the skin of the patient, but is not limited thereto.

In some embodiments, the ECU 602 can include a switching network configured to interchange which of electrodes of a catheter (e.g., 502) are coupled to which portions of the ECU. In some such embodiments, a user can manually switch which inputs receive connections to which electrodes of the catheter 502. Such configurability allows for a system operator to adjust the direction of propagation of the elicited potential as desired. For example, the switching network, or more generally switches, can be used to connect the electrodes 524 and 525 to the stimulator 606 during a period of time during which electrical stimulation pulses are to be emitted by the catheter 502, and the switches can be used to connect the electrodes 526 and 526 to the amplifier 612 to sense the elicited response to the electrical stimulation pulses. Additionally, or alternatively, a controller (e.g., 622) can autonomously control such a switching network.

The amplifier 612 can include any appropriate amplifier for amplifying desired signals or attenuating undesired signals. In some examples, the amplifier has a high common-mode rejection ratio (CMRR) for eliminating or substantially attenuating undesired signals present at each of the sense electrodes (e.g., 526 and 527). In some embodiments, the amplifier 612 can be adjusted, for example, via an adjustable capacitance or via other attributes of the amplifier.

In the example system 600 of FIG. 6, the ECU 602 further includes a filter 614 for enhancing the desired signal in the signal received via a pair of the electrodes. The filter 614 can include a band-pass filter, a notch filter, or any other appropriate filter to isolate desired signals from noise artifacts within the received signals. In some embodiments, various properties of the filter 614 can be adjusted to manipulate its filtering characteristics. For example, the filter may include an adjustable capacitance or other parameter to adjust its frequency response.

At least one of amplification and filtering of a sensed signal (e.g., received at the electrodes 526 and 527) can allow for extraction of the desired signal at 616. In some embodiments, extraction 616 comprises at least one additional processing step to isolate desired signals from the signal sensed using electrodes such as preparing the signal for output at 618. In some embodiments, the functionalities of any combination of amplifier 612, filter 614, and extraction 616 may be combined into a single entity. For instance, the amplifier 612 may act to filter undesired frequency content from the signal without requiring additional filtering at a separate filter.

In some embodiments, the ECU 602 can record emitted stimuli and/or received signals. Such data can be subsequently stored in permanent or temporary memory 620. The ECU 602 can comprise such memory 620 or can otherwise be in communication with external memory (not shown). Thus, the ECU 602 can be configured to emit stimulus pulses to electrodes of the catheter, record such pulses in a memory, receive signals from the catheter, and also record such received signal data. The memory 620 in or associated with the ECU 602 can be internal or external to any part of the ECU 602 or the ECU 602 itself.

The ECU 602 or separate external processor can further perform calculations on the stored data to determine characteristics of signals either emitted or received via the catheter. For example, in various embodiments, the ECU 602 can determine any of the amplitude, duration, or timing of occurrence of the received or emitted signals. The ECU 602 can further determine the relationship between the received signal and the emitted stimulus signal, such as a temporal relationship therebetween. In some embodiments, the ECU 602 performs signal averaging on the signal data received from the catheter. Such averaging can act to reduce random temporal noise in the data while strengthening the data corresponding to any elicited potentials received by the catheter.

Averaging as such can result in a signal in which temporally random noise is generally averaged out and the signal present in each recorded data set, such as elicited potentials, will remain high. In some embodiments, each iteration of the process can include a synchronization step so that each acquired data set can be temporally registered to facilitate averaging the data. That is, events that occur consistently at the same time during each iteration may be detected, while temporally random artifacts (e.g., noise) can be reduced. In general, the signal to noise ratio (SNR) resulting in such averaging will improve by the square root of the number of samples averaged in order to create the averaged data set.

The ECU 602 can further present information regarding any or all of the applied stimulus, the signal, and the results of any calculations to a user of the system, e.g., via output 618. For example, the ECU 602 can generate a graphical display providing one or more graphs of signal strength vs. time representing the stimulus and/or the received signal.

In some embodiments, the ECU 602 can include a controller 622 in communication with one or both of stimulator 606 and SENS subsystem 604. The controller 622 can be configured to cause stimulator 606 to apply a stimulation signal to a catheter, e.g., the catheter 502. Additionally or alternatively, the controller 622 can be configured to analyze signals received and/or output by the SENS subsystem 604. In some embodiments, the controller 622 can act to control the timing of applying the stimulation signal from stimulator 606 and the timing of receiving signals by the SENS subsystem 604. The controller 622 can be implemented, e.g., using one or more processors, field programmable gate arrays (FPGAs), state machines, and/or application specific integrated circuits (ASICs), but is not limited thereto.

Example electrical control units have been described. In various embodiments, the ECU 602 can emit stimulus pulses to the catheter 502, receive signals from the catheter 502, perform calculations on the emitted and/or received signals, and present the signals and/or results of such calculations to a user. In some embodiments, the ECU 602 can comprise separate modules for emitting, receiving, calculating, and providing results of calculations. Additionally or alternatively, the functionality of controller 622 can be integrated into the ECU 602 as shown, or can be separate from and in communication with the ECU.

The controller 622 can also control an optional fluid supply subsystem 628, which can include a cartridge and a reservoir. The fluid supply subsystem 628 can be fluidically coupled to one or more fluid lumens within the catheter shaft 522 which in turn are fluidically coupled to the balloon 513. The fluid supply subsystem 628 can be configured to circulate a cooling liquid through the catheter 502 to the transducer 511 in the balloon 513.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited.

What is claimed is:

1. A method for use in evaluating neural electrical activity of renal nerves in tissue surrounding a renal artery of a patient for which a renal denervation procedure has been or is going to be performed, the method comprising:
inserting a distal portion of a guide catheter through an abdominal aorta of a patient such that the distal portion of the guide catheter is positioned adjacent to a renal artery ostium located along a lateral aspect of the abdominal aorta, or such that the distal portion of the guide catheter is inserted through the renal artery ostium and into a proximal portion of the renal artery of the patient;
using the guide catheter to insert a distal portion of a mapping catheter through a distal most end of the guide catheter such that the distal portion of the mapping catheter is positioned within the renal artery of the patient;
while the distal portion of the mapping catheter is positioned within the renal artery of the patient,
using a stimulation electrode positioned in the abdominal aorta within 10 millimeters (mm) of the renal artery ostium to deliver one or more electrical stimulation pulses to thereby evoke a neural electrical response from at least some of the renal nerves in tissue surrounding the renal artery, wherein the stimulation electrode is located on the mapping catheter, on the distal portion of the guide catheter, or on a guidewire; and
using a sense electrode positioned in the renal artery, downstream from the stimulation electrode, to sense the neural electrical activity evoked in response to the one or more electrical stimulation pulses, wherein the sense electrode is located on the mapping catheter; and
using the sensed neural electrical activity, evoked in response to the one or more electrical stimulation pulses, as a pre-denervation or post-denervation measurement associated with a renal denervation procedure.

2. The method of claim 1, wherein the using the sensed neural electrical activity, evoked in response to the one or more electrical stimulation pulses, as the pre-denervation or post-denervation measurement associated with the renal denervation procedure, comprises using the sensed neural electrical activity to select one or more renal denervation parameters and performing renal denervation using the one or more renal denervation parameters selected.

3. The method of claim 1, wherein the sense electrode, which is used to sense the neural electrical activity evoked in response to the one or more electrical stimulation pulses, is positioned downstream of the stimulation electrode by at least 1.5 centimeters.

4. The method of claim 1, wherein the sense electrode, which is used to sense the neural electrical activity evoked in response to the one or more electrical stimulation pulses, is positioned downstream of the stimulation electrode by at least 2.0 centimeters.

5. The method of claim 1, wherein the sense electrode, which is used to sense the neural electrical activity evoked in response to the one or more electrical stimulation pulses, is positioned downstream of the stimulation electrode by at least 3.0 centimeters.

6. The method of claim 1, wherein the one or more electrical stimulation pulses are delivered between the stimulation electrode and a return electrode that is also positioned in the abdominal aorta within 10 mm of the renal artery ostium.

7. The method of claim 1, wherein the one or more electrical stimulation pulses are delivered between the stimulation electrode and a return surface electrode that is positioned on skin of the patient.

8. The method of claim 1, wherein the stimulation electrode comprises a deployable stimulation electrode that is deployed such that an outer circumference of the deployable stimulation electrode is in contact with an inner wall of the abdominal aorta within 10 mm of the renal artery ostium.

9. The method of claim 8, wherein the sense electrode comprises a deployable sense electrode that is deployed such that an outer circumference of the deployable sense electrode is in contact with an inner wall of the renal artery, downstream of the deployable stimulation electrode.

10. The method of claim 9, wherein the deployable sense electrode is used together with another sense electrode to sense the neural electrical activity evoked in response to the one or more electrical stimulation pulses, wherein the other sense electrode is also located on the mapping catheter downstream of the stimulation electrode or is a surface electrode that is positioned on skin of the patient.

11. A method for use in evaluating neural electrical activity of renal nerves in tissue surrounding a renal artery of a patient for which a renal denervation procedure has been or is going to be performed, the method comprising:
inserting a distal portion of a guide catheter through an abdominal aorta of a patient such that the distal portion of the guide catheter is positioned adjacent to a renal artery ostium located along a lateral aspect of the abdominal aorta, or such that the distal portion of the guide catheter is inserted through the renal artery ostium and into a proximal portion of the renal artery of the patient;
using the guide catheter to insert a distal portion of a mapping catheter through a distal most end of the guide catheter such that the distal portion of the mapping catheter is positioned within the renal artery of the patient;
while the distal portion of the mapping catheter is positioned within the renal artery of the patient,
using a stimulation electrode positioned in the abdominal aorta to deliver one or more electrical stimulation pulses to an aorticorenal ganglia thereby evoking a neural electrical response from at least some of the renal nerves in tissue surrounding the renal artery, wherein the stimulation electrode is located on the mapping catheter, on the distal portion of the guide catheter, or on a guidewire; and
using a sense electrode positioned in the renal artery, downstream from the stimulation electrode, to sense the neural electrical activity evoked in response to the one or more electrical stimulation pulses, wherein the sense electrode is located on the mapping catheter; and
using the sensed neural electrical activity, evoked in response to the one or more electrical stimulation pulses, as a pre-denervation or post-denervation measurement associated with a renal denervation procedure.

12. The method of claim 11, wherein the using the sensed neural electrical activity, evoked in response to the one or more electrical stimulation pulses, as the pre-denervation or post-denervation measurement associated with the renal denervation procedure, comprises using the sensed neural electrical activity to select one or more renal denervation parameters and performing renal denervation using the one or more renal denervation parameters selected.

13. The method of claim 11, wherein the sense electrode, which is used to sense the neural electrical activity evoked in response to the one or more electrical stimulation pulses, is positioned downstream of the stimulation electrode by at least 1.5 centimeters.

14. The method of claim 11, wherein the sense electrode, which is used to sense the neural electrical activity evoked in response to the one or more electrical stimulation pulses, is positioned downstream of the stimulation electrode by at least 2.0 centimeters.

15. The method of claim 11, wherein the sense electrode, which is used to sense the neural electrical activity evoked in response to the one or more electrical stimulation pulses, is positioned downstream of the stimulation electrode by at least 3.0 centimeters.

16. The method of claim 11, wherein the one or more electrical stimulation pulses are delivered between the stimulation electrode and a return electrode that is also positioned in the abdominal aorta within 10 mm of a renal artery ostium of the patient.

17. A method for use in evaluating neural electrical activity of renal nerves in tissue surrounding a renal artery of a patient for which a renal denervation procedure has been or is going to be performed, the method comprising:
using a stimulation electrode positioned in an abdominal aorta of the patient to deliver one or more electrical stimulation pulses to an aorticorenal ganglia thereby evoking a neural electrical response from at least some of the renal nerves in tissue surrounding the renal artery;
using a sense electrode positioned in the renal artery, downstream from the stimulation electrode, to sense the neural electrical activity evoked in response to the one or more electrical stimulation pulses; and
using the sensed neural electrical activity, evoked in response to the one or more electrical stimulation pulses, as a pre-denervation or post-denervation measurement associated with a renal denervation procedure.

18. The method of claim 17, wherein the using the sensed neural electrical activity, evoked in response to the one or more electrical stimulation pulses, as the pre-denervation or post-denervation measurement associated with the renal denervation procedure, comprises using the sensed neural electrical activity to select one or more renal denervation parameters and performing renal denervation using the one or more renal denervation parameters selected.

19. The method of claim 17, wherein the sense electrode, which is used to sense the neural electrical activity evoked in response to the one or more electrical stimulation pulses, is positioned downstream of the stimulation electrode by at least 1.5 centimeters.

20. The method of claim 17, wherein the sense electrode, which is used to sense the neural electrical activity evoked in response to the one or more electrical stimulation pulses, is positioned downstream of the stimulation electrode by at least 2.0 centimeters.

21. The method of claim 17, wherein the sense electrode, which is used to sense the neural electrical activity evoked in response to the one or more electrical stimulation pulses, is positioned downstream of the stimulation electrode by at least 3.0 centimeters.

22. The method of claim 17, wherein the one or more electrical stimulation pulses are delivered between the stimulation electrode and a return electrode that is also positioned in the abdominal aorta within 10 mm of a renal artery ostium of the patient.
